(19) United States
(12) Patent Application Publication
(10) Pub. No.: US 2012/0149741 A1
(43) Pub. Date: Jun. 14, 2012

(54) TREATMENT OF DUCHENNE MUSCULAR DYSTROPHY

(75) Inventor: Graham Michael WYNNE, (US)

(73) Assignees: Biomarin IGA Limited, Nassau (BS); Summit Corporation PLC, Oxfordshire (GB)

(21) Appl. No.: 12/599,967

(22) PCT Filed: Aug. 14, 2008
(86) PCT No.: PCT/EP08/06717

§ 371 Date: Apr. 21, 2011

(30) Foreign Application Priority Data
Aug. 15, 2007 (GB) 0715938.7

(51) Int. Cl. A61K 31/416; A61P 21/00; A61K 31/4192; C07D 231/56; C07D 249/20

(52) U.S. Cl. 514/359; 548/361.1; 514/406; 548/259

(57) ABSTRACT

There are provided compounds of Formula (I) wherein three of A¹, A², A³, and A⁴ represent CH and one of A¹, A², A³, and A⁴ represents CR¹; R¹ represents SO₂R2 or NHCOR2 wherein R2 represents C₁-C₆ alkyl optionally substituted by one or more halogen, hydroxyl or alkoxy groups; R⁹ represents C₆-C₁₀ aryl; or a pharmaceutically acceptable salt thereof.

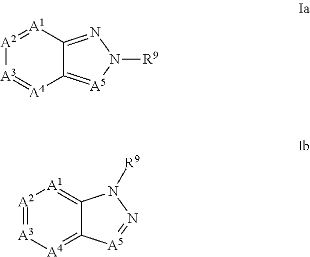

### RELATED APPLICATION

Priority is claimed herein to GB0715938.7, filed Aug. 15, 2007, entitled “TREATMENT OF DUCHENNE MUSCULAR DYSTROPHY.” The disclosure of the above-referenced application is incorporated by reference in its entirety.

### FIELD

Provided herein is a method of treatment of Duchenne muscular dystrophy.

### BACKGROUND

Duchenne muscular dystrophy (DMD) is a common, genetic neuromuscular disease associated with the progressive deterioration of muscle function, first described over 150 years ago by the French neurologist, Duchenne de Boulogne, after whom the disease is named. DMD has been characterized as an X-linked recessive disorder that affects 1 in 3,500 males caused by mutations in the dystrophin gene. The gene is the largest in the human genome, encompassing 2.6 million base pairs of DNA and containing 79 exons. Approximately 60% of dystrophin mutations are large insertion or deletions that lead to frameshift errors downstream, whereas approximately 40% are point mutations or small frameshift rearrangements. The vast majority of DMD patients lack the dystrophin protein. Becker muscular dystrophy is a much milder form of DMD caused by reduction in the amount, or alteration in the size, of the dystrophin protein. The high incidence of DMD (1 in 10,000 sperm or eggs) means that genetic screening will never eliminate the disease, so an effective therapy is highly desirable.

A number of natural and engineered animal models of DMD exist, and provide a mainstay for preclinical studies (Allamand, V. & Campbell, K. P. Animal models for muscular dystrophy: valuable tools for the development of therapies. Hum. Mol. Genet. 9, 2459-2467 (2000). Although the mouse, cat and dog models all have mutations in the DMD gene and exhibit a biochemical dystrophinopathy similar to that seen in humans, they show surprising and considerable variation in terms of their phenotype. Like humans, the canine (Golden retriever muscular dystrophy and German short-haired pointer) models have a severe phenotype; these dogs typically die of cardiac failure. Dogs offer the best phenocopy for human disease, and are considered a high benchmark for preclinical studies. Unfortunately, breeding these animals is expensive and difficult, and the clinical time course can be variable among litters.

The mdx mouse is the most widely used model due to availability, short gestation time, time to mature and relatively low cost (Bulfield, G., Siller, W. G., Wight, P. A. & Moore, K. J. X chromosome-linked muscular dystrophy (mdx) in the mouse. Proc. Natl. Acad. Sci. USA 81, 1189-1192 (1984)).

Since the discovery of the DMD gene about 20 years ago, varying degrees of success in the treatment of DMD have been achieved in preclinical animal studies, some of which are being followed up in humans. Present therapeutic strategies can be broadly divided into three groups: first, gene therapy approaches; second, cell therapy; and last, pharmacological therapy. Gene- and cell-based therapies offer the fundamental advantage of obviating the need to separately correct secondary defects/pathology (for example, contractures), especially if initiated early in the course of the disease. Unfortunately, these approaches face a number of technical hurdles. Immunological responses against viral vectors, myoblasts and newly synthesized dystrophin have been reported, in addition to toxicity, lack of stable expression and difficulty in delivery.

Pharmacological approaches for the treatment of muscular dystrophy differ from gene- and cell-based approaches in not being designed to deliver either the missing gene and/or protein. In general, the pharmacological strategies use drugs/molecules in an attempt to improve the phenotype by means such as decreasing inflammation, improving calcium homeostasis and increasing muscle progenitor proliferation or commitment. These strategies offer the advantage that they are easy to deliver systemically and can circumvent many of the immunological and/or toxicity issues that are related to vectors and cell-based therapies. Although investigations with corticosteroids and sodium cromoglycate, to reduce inflammation, dantrolene to maintain calcium homeostasis and clenbuterol to increase muscle strength, have produced promising results none of these potential therapies has yet been shown to be effective in treating DMD.

An alternative pharmacological approach is upregulation therapy. Upregulation therapy is based on increasing the expression of alternative genes to replace a defective gene and is particularly beneficial when an immune response is mounted against a previously absent protein. Upregulation of utrophin, an autosomal paralogue of dystrophin has been proposed as a potential therapy for DMD (Perkins & Davies, Neuromuscul Disord, S1: S78-S89 (2002), Khurana & Davies, Nat Rev Drug Discov 2:379-390 (2003)). When utrophin is overexpressed in transgenic mdx mice it localizes to the sarcolemma of muscle cells and restores the components of the dystrophin-associated protein complex (DAPC), which prevents the dystrophic development and in turn leads to functional improvement of skeletal muscle. Adenoviral delivery of utrophin in the dog has been shown to prevent pathology. Commencement of increased utrophin expression shortly after birth in the mouse model can be effective and no toxicity is observed when utrophin is ubiquitously expressed, which is promising for the translation of this therapy to humans. Upregulation of endogenous utrophin to sufficient levels to decrease pathology might be achieved by the delivery of small diffusible compounds.

### DESCRIPTION

Compounds which upregulate endogenous utrophin in predictive screens and, thus, may be useful in the treatment of DMD are provided.

Provided are compounds of Formula I

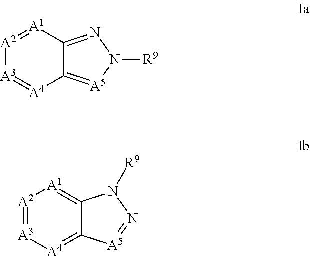

wherein
three of A¹, A², A³, and A⁴ represent CH and one of A¹, A², A³, and A⁴ represents CR¹;
R¹ represents SO₂R² or NHCOR² wherein R² represents C₁-C₆ alkyl optionally substituted by one or more halogen, hydroxyl or alkoxy groups (C₁-C₆ alkoxy groupy);
R⁹ represents aryl optionally substituted by one or more halogen groups;
or a pharmaceutically acceptable salt thereof.

Also provided is the use of the compounds of formula I in the manufacture of a medicament for the therapeutic and/or prophylactic treatment of Duchenne muscular dystrophy, Becker muscular dystrophy or cachexia.

Compounds of formula I may exist in tautomeric, enantiomeric and diastereomeric forms, all of which are included within the scope of this disclosure.

The instant disclosure will now be described with reference to the accompanying drawings in which:

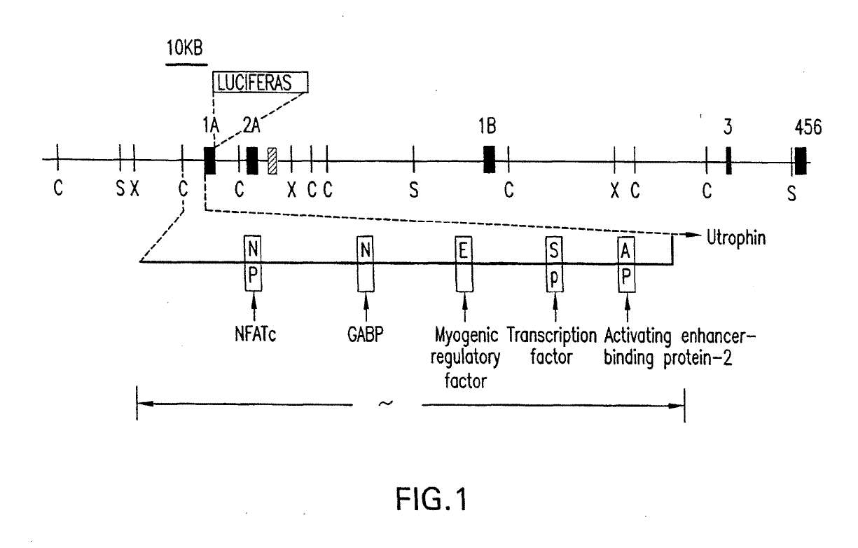

FIG. 1 shows a luciferase reporter assay (murine H2K cells).

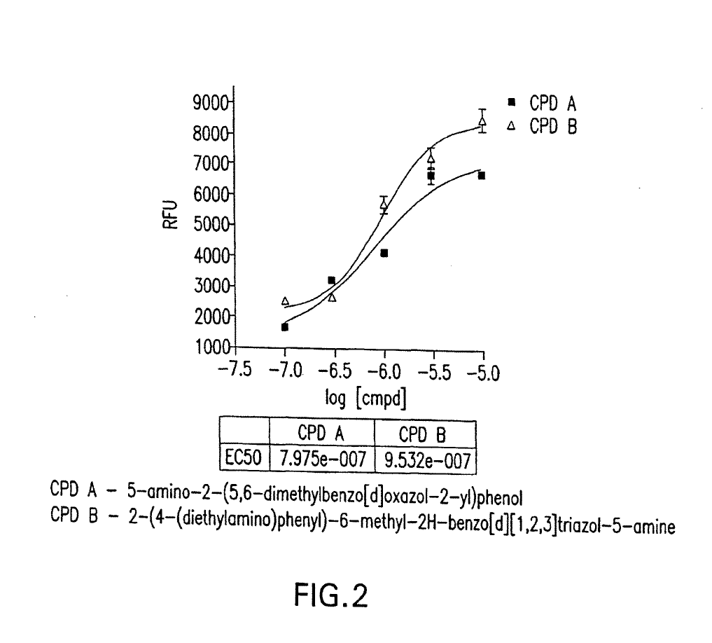

FIG. 2 shows a dose dependent luciferase induction.

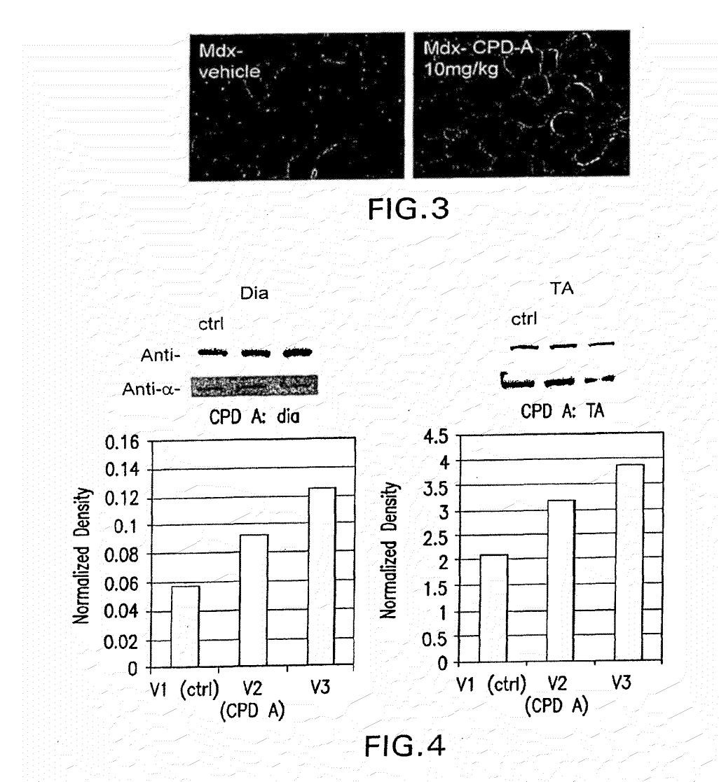

FIG. 3 shows an example of TA muscle sections stained with antibody specific for mouse utrophin.

FIG. 4 shows that mice exposed to CPD-A (V2 and V3) showed increased levels of utrophin expression compared to control.

All of the compounds of formula I may be made by conventional methods. Methods of making heteroaromatic ring systems are well known in the art. In particular, methods of synthesis are discussed in Comprehensive Heterocyclic Chemistry, Vol. 1 (Eds.: A R Katritzky, C W Rees), Pergamon Press, Oxford, 1984 and Comprehensive Heterocyclic Chemistry II: A Review of the Literature 1982-1995 The Structure, Reactions, Synthesis, and Uses of Heterocyclic Compounds, Alan R. Katritzky (Editor), Charles W. Rees (Editor), E. F. V. Scriven (Editor), Pergamon Pr, June 1996. Other general resources which would aid synthesis of the compounds of interest include March's Advanced Organic Chemistry: Reactions, Mechanisms, and Structure, Wiley-Interscience; 5th edition (Jan. 15, 2001).

Compounds of formula I or pharmaceutically acceptable salts thereof may be prepared from a compound of formula II

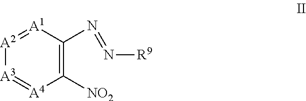

in which A¹, A², A³, and A⁴ are defined as above, in a reductive ring closure effected by reaction with thiourea-S,S-dioxide or a dithionite salt, for example an alkali metal salt, as described, for example, in EP 0 751 134. The reaction may be carried out in an aqueous solution, in another embodiment an alcoholic aqueous solution, at a temperature of 60 to 80° C. Cyclisation will not occur in the presence of certain functionality, for example in the presence of —NH₂ or —OH functionality. These groups will need to be protected before cyclisation. For example —NH₂ groups may be protected as amides, and OH groups may be protected as ethers. Suitable protecting strategies are disclosed, for example, in EP 0 751 134.

Compounds of formula II may be prepared by a diazonium coupling reaction of a diazonium compound of formula III,

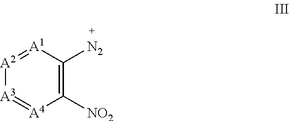

wherein A¹, A², A³, and A⁴ are defined as above, with phenyl derivatives of formula IV

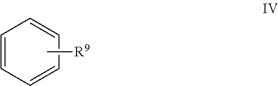

wherein R⁹ is defined as above. Conditions for the coupling are well known to the synthetic chemist. For example, reaction may take place in methanol under slightly acidic conditions, over up to 24 hours.

Compounds of formula III may be prepared by diazotisation of appropriate amines of formula V

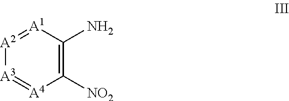

wherein A¹, A², A³, and A⁴ are defined as above. Methods of diazotisation are well known in the art, e.g. by reaction with NaNO₂/AcOH in an aqueous solution at 0 to 10° C.

Compounds of formula V may be synthesised by nitration, and subsequent deprotection, of a compound of formula VI,

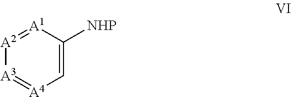

wherein A¹, A², A³, and A⁴ are as defined above and P represents a protecting group appropriate to the nitrating conditions. Nitration could be effected by, for example, cHNO₃/CH₂SO₄ in a solvent appropriate to the reaction conditions.

Compounds of formulas IV and VI may be made by conventional techniques known per se.

In the above processes it may be necessary for any functional groups, e.g. hydroxy or amino groups, present in the starting materials to be protected, thus it may be necessary to remove one or more protective groups to generate the compound of formula I.

Suitable protecting groups and methods for their removal are, for example, those described in “Protective Groups in Organic Synthesis” by T. Greene and P. G. M. Wutts, John Wiley and Sons Inc., 1991. Hydroxy groups may, for example, be protected by arylmethyl groups such as phenylmethyl, diphenylmethyl or triphenylmethyl; acyl groups such as acetyl, trichloroacetyl or trifluoroacetyl; or as tetrahydropyranyl derivatives. Suitable amino protecting groups include arylmethyl groups such as benzyl, (R,S)-α-phenylethyl, diphenylmethyl or triphenylmethyl, and acyl groups such as acetyl, trichloroacetyl or trifluoroacetyl. Conventional methods of deprotection may be used including hydrogenolysis, acid or base hydrolysis, or photolysis. Arylmethyl groups may, for example, be removed by hydrogenolysis in the presence of a metal catalyst e.g. palladium on charcoal. Tetrahydropyranyl groups may be cleaved by hydrolysis under acidic conditions. Acyl groups may be removed by hydrolysis with a base such as sodium hydroxide or potassium carbonate, or a group such as trichloroacetyl may be removed by reduction with, for example, zinc and acetic acid.

The compounds of formula I, and salts thereof, may be isolated from their reaction mixtures using conventional techniques.

Salts of the compounds of formula I may be formed by reacting the free acid, or a salt thereof, or the free base, or a salt or derivative thereof, with one or more equivalents of the appropriate base or acid. The reaction may be carried out in a solvent or medium in which the salt is insoluble or in a solvent in which the salt is soluble, e.g. ethanol, tetrahydrofuran or diethyl ether, which may be removed in vacuo, or by freeze drying. The reaction may also be a metathetical process or it may be carried out on an ion exchange resin.

Pharmaceutically acceptable salts of the compounds of formula I include alkali metal salts, e.g. sodium and potassium salts; alkaline earth metal salts, e.g. calcium and magnesium salts; salts of the Group III elements, e.g. aluminium salts; and ammonium salts. Salts with suitable organic bases, for example, salts with hydroxylamine; lower alkylamines, e.g. methylamine or ethylamine; with substituted lower alkylamines, e.g. hydroxy substituted alkylamines; or with monocyclic nitrogen heterocyclic compounds, e.g. piperidine or morpholine; and salts with amino acids, e.g. with arginine, lysine etc, or an N-alkyl derivative thereof; or with an aminosugar, e.g. N-methyl-D-glucamine or glucosamine. In one embodiment, the salts are non-toxic physiologically acceptable salts, although other salts are also useful, e.g. in isolating or purifying the product.

Diastereoisomers may be separated using conventional techniques, e.g. chromatography or fractional crystallisation. The various optical isomers may be isolated by separation of a racemic or other mixture of the compounds using conventional, e.g. fractional crystallisation or HPLC, techniques. Alternatively the desired optical isomers may be made by reaction of the appropriate optically active starting materials under conditions which will not cause racemisation.

Substituents that alkyl may represent include methyl, ethyl, butyl, and sec butyl.

Substituents that aryl may represent include C₅-C₁₀ carbocycles, which may be mono- or bicyclic, and partially or fully aromatic, optionally substituted by one or more halogens.

Halogen may represent F, Cl, Br and I.

Also provided is a method for the treatment or prophylaxis of Duchenne muscular dystrophy, Becker muscular dystrophy or cachexia in a patient in need thereof, comprising administering to the patient an effective amount of a compound of formula (I) or a pharmaceutically acceptable salt thereof.

In another embodiment, the compounds have formula Ia, wherein:

three of A¹, A², A³, and A⁴ represent CH and one of A¹, A², A³, and A⁴ represents CR¹ wherein R¹ represents SO₂R² or NHCOR² wherein R² represents C₁-C₆ alkyl optionally substituted by one or more halogen, hydroxyl or C₁₋₆ alkoxy groups;
and R⁹ represents C₆-C₁₀ aryl.

In another embodiment, in the above defined group of compounds R⁹ represents 2-naphthyl or 4-chlorophenyl.

In another embodiment, A¹, A² and A⁴ represent CH and A³ represents CR¹.

In another embodiment, R² is ethyl or isopropyl.

The compounds of formula I for use in the treatment of DMD will generally be administered in the form of a pharmaceutical composition.

Thus, according to a further aspect of this disclosure there is provided a pharmaceutical composition including in one embodiment less than 80% w/w, in another embodiment less than 50% w/w, e.g. 0.1 to 20%, of a compound of formula I, or a pharmaceutically acceptable salt thereof, as defined above, in admixture with a pharmaceutically acceptable diluent or carrier.

Also provided is a process for the production of such a pharmaceutical composition which comprises mixing the ingredients. Examples of pharmaceutical formulations which may be used, and suitable diluents or carriers, are as follows:

for intravenous injection or infusion—purified water or saline solution;

for inhalation compositions—coarse lactose;

for tablets, capsules and dragees—microcrystalline cellulose, calcium phosphate, diatomaceous earth, a sugar such as lactose, dextrose or mannitol, talc, stearic acid, starch, sodium bicarbonate and/or gelatin;

for suppositories—natural or hardened oils or waxes.

When the compound is to be used in aqueous solution, e.g. for infusion, it may be necessary to incorporate other excipients. In particular there may be mentioned chelating or sequestering agents, antioxidants, tonicity adjusting agents, pH-modifying agents and buffering agents.

Solutions containing a compound of formula I may, if desired, be evaporated, e.g. by freeze drying or spray drying, to give a solid composition, which may be reconstituted prior to use.

When not in solution, the compound of formula I, in one embodiment, is in a form having a mass median diameter of from 0.01 to 10 μm. The compositions may also contain suitable preserving, stabilising and wetting agents, solubilisers, e.g. a water-soluble cellulose polymer such as hydroxypropyl methylcellulose, or a water-soluble glycol such as propylene glycol, sweetening and colouring agents and flavourings. Where appropriate, the compositions may be formulated in sustained release form.

The content of compound formula I in a pharmaceutical composition is generally about 0.01-about 99.9 wt %, in one embodiment about 0.1-about 50 wt %, relative to the entire preparation.

The dose of the compound of formula I is determined in consideration of age, body weight, general health condition, diet, administration time, administration method, clearance rate, combination of drugs, the level of disease for which the patient is under treatment then, and other factors.

While the dose varies depending on the target disease, condition, subject of administration, administration method and the like, for oral administration as a therapeutic agent for the treatment of Duchenne muscular dystrophy in a patient suffering from such a disease is from 0.01 mg-10 g, in one embodiment 0.1-100 mg, is in one embodiment administered in a single dose or in 2 or 3 portions per day.

The potential activity of the compounds of formula I for use in the treatment of DMD may be demonstrated in the following predictive assay and screens.

### 1. Luciferase Reporter Assay (Murine H2K Cells)

The cell line used for the screen is an immortalized mdx mouse H2K cell line that has been stably transfected with a plasmid containing fragment of the Utrophin A promoter including the first untranslated exon linked to a luciferase reporter gene (see FIG. 1).

Under conditions of low temperature and interferon containing media, the cells remain as myoblasts. These are plated into 96 well plates and cultured in the presence of compound for three days. The level of luciferase is then determined by cell lysis and reading of the light output from the expressed luciferase gene utilising a plate luminometer.

Example of pharmacological dose response of compounds in the assay is shown in FIG. 2

### 2. mdx Mouse

Data obtained from the ADMET data was prioritised and the compounds with the best in vitro luciferase activity and reasonable ADMET data were prioritised for testing in the mdx proof of concept study where the outcome was to identify whether any of the compounds had the ability to increase the levels of utrophin protein in dystrophin deficient muscle when compared to vehicle only dosed control animals.

There were two animals injected with 10 mg/kg of compound administered ip daily for 28 days plus age matched controls. Muscle samples were taken and processed for sectioning (to identify increases in sarcolemmal staining of utrophin) and Western blotting (to identify overall increases in utrophin levels).

FIG. 3. shows an example of TA muscle sections stained with antibody specific for mouse utrophin. Comparison to the mdx muscle only injected with vehicle shows an increase in the amount of sarcolemmal bound utrophin.

Muscles from the above treated mice were also excised and processed for Western blotting and stained with specific antibodies (see FIG. 4). Again using muscle dosed with CPD-A shows a significant increase in the overall levels of utrophin present in both the TA leg muscle and the diaphragm. Both mice exposed to CPD-A (V2 and V3) showed increased levels of utrophin expression compared to control.

Positive upregulation data from the first 28 day study were then repeated in a further two mouse 28 day study. A total of three different compounds have shown in duplicate the ability to increase the level of utrophin expression in the mdx mouse when delivered daily by ip for 28 days. This data demonstrates the ability of the compound when delivered ip causes a significant increase in the levels of utrophin found in the mdx muscle and therefore gives us the confidence that this approach will ameliorate the disease as all the published data to date demonstrates that any increase of utrophin levels over three fold has significant functional effects on dystrophin deficient muscle.

### The H2K/mdx/Utro A Reporter Cell Line Maintenance

The H2K/mdx/Utro A reporter cell line was passaged twice a week until ≦30% confluent. The cells were grown at 33° C. in the presence of 10% CO₂.

To remove the myoblasts for platting, they were incubated with Trypsin/EDTA until the monolayer started to detach.

### Growth Medium

DMEM Gibco 41966
20% FCS
1% Pen/Strep
1% glutamine
10 mls Chick embryo extract
Interferon (1276 905 Roche) Add fresh 10 μl/50 mls medium

### Luciferase Assay for 96 Well Plates

The H2K/mdx/Utro A reporter cell line cells were plated out into 96 well plates (Falcon 353296, white opaque) at a density of approximately 5000 cells/well in 190 μl normal growth medium. The plates were then incubated at 33° C. in the presence of 10% CO₂ for 24 hrs.

Compounds were dosed by adding 10 μl of diluted compound to each well giving a final concentration of 10 μM. The plates were then incubated for a further 48 hrs.

Cells were then lysed in situ following the manufacture's protocols (Promega Steady-Glo Luciferase Assay System (E2520), then counted for 10 seconds using a plate luminometer (Victor1420).

### Compound Storage

Compounds for screening were stored at −20° C. as 10 mM stocks in 100% DMSO until required.

Injection of mdx Mice with Compounds

Mdx from a breeding colony were selected for testing. Mice were injected daily with either vehicle or 10 mg/kg of compound using the intreperitoneal route (ip). Mice were weighed and compounds diluted in 5% DMSO, 0.1% tween in PBS.

Mice were sacrificed by cervical dislocation at desired time points, and muscles excised for analysis.

### Muscle Analysis

### Immunohistochemistry

Tissues for sectioning were dissected, immersed in OCT (Bright Cryo-M-Bed) and frozen on liquid nitrogen cooled isopentane. Unfixed 8 μM cryosections were cut on a Bright Cryostat, and stored at −80° C.

In readiness for staining, sections were blocked in 5% fetal calf serum in PBS for 30 mins. The primary antibodies were diluted in blocking reagent and incubated on sections for 1.5 hrs in a humid chamber then washed three times for 5 mins in PBS. Secondary antibodies were also diluted in blocking reagent, and incubated for 1 hr in the dark in a humid chamber. Finally sections were washed three times 5 mins in PBS and coverslips were mounted with hydromount. Slides were analysed using a Leica fluorescent microscope.

### Results

Biological activity was assessed using the luciferase reporter assay in murine H2K cells, and is classified as follows:

+ Up to 200% relative to control
++ Between 201% and 300% relative to control
+++ Between 301% and 400% relative to control
++++ Above 401% relative to control

TABLE 1
Compounds made by methods described herein

| Example | | |
|---|---|---|
| number | Chemical Name | Activity |
| 1 | N-(1-(naphthalen-2-yl)-1H-indazol-6- | + |
| | yl)isobutyramide | |
| 2 | N-(2-(naphthalen-2-yl)-2H-indazol-6- | ++++ |
| | yl)isobutyramide | |
| 3 | 2-(4-chlorophenyl)-6-(ethylsulfonyl)-2H- | ++ |
| | indazole | |
| 4 | 5-(ethylsulfonyl)-2-(naphthalen-2′-yl)-2H- | ++ |
| | benzo[d][1,2,3]triazole | |

### EXAMPLES

HPLC-UV-MS was performed on a Gilson 321 HPLC with detection performed by a Gilson 170 DAD and a Finnigan AQA mass spectrometer operating in electrospray ionisation mode. The HPLC column used is a Phenomenex Gemini C18 150×4.6 mm. Preparative HPLC was performed on a Gilson 321 with detection performed by a Gilson 170 DAD. Fractions were collected using a Gilson 215 fraction collector. The preparative HPLC column used is a Phenomenex Gemini C18 150×10 mm and the mobile phase is acetonitrile/water.

¹H NMR spectra were recorded on a Bruker instrument operating at 300 MHz. NMR spectra were obtained as CDCl₃ solutions (reported in ppm), using chloroform as the reference standard (7.25 ppm) or DMSO-D₆ (2.50 ppm). When peak multiplicities are reported, the following abbreviations are used s (singlet), d (doublet), t (triplet), m (multiplet), br (broadened), dd (doublet of doublets), dt (doublet of triplets), td (triplet of doublets).

Coupling constants, when given, are reported in Hertz (Hz).

Column chromatography was performed either by flash chromatography (40-65 μm silica gel) or using an automated purification system (SP1™ Purification System from Biotage). Reactions in the microwave were done in an Initiator 8™ (Biotage).

The abbreviations used are DMSO (dimethylsulfoxide), HCl (hydrochloric acid), MgSO₄ (magnesium sulfate), NaOH (sodium hydroxide), Na₂CO₃ (sodium carbonate), NaHCO₃ (sodium bicarbonate), THF (tetrahydrofuran).

### Example 1

### N-(2-(Naphthalen-2-yl)-2H-indazol-6-yl)isobutyramide and N-(1-(naphthalen-2-yl)-1H-indazol-6-yl)isobutyramide

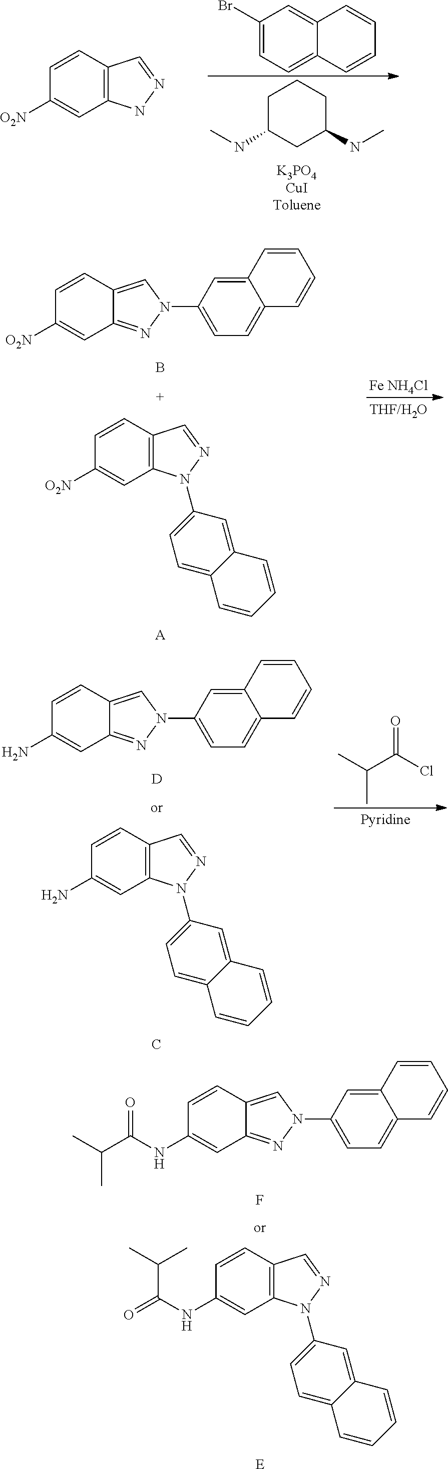

### Step 1: Arylation

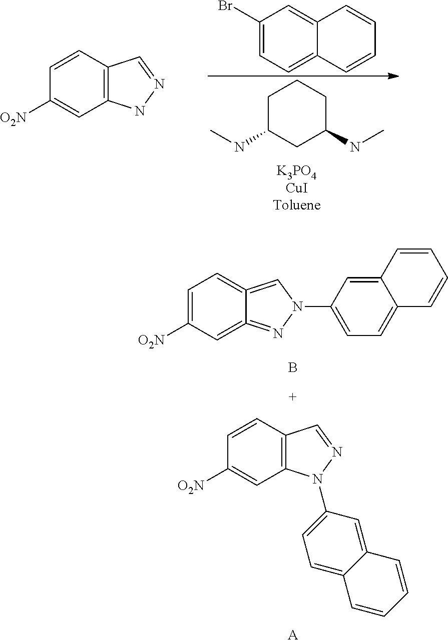

To a seal tube dried in the oven were added copper iodide (29.2 mg, 0.05 eq, 0.153 mmol), 6-nitroindazole (500 mg, 1 eq, 3 mmol) and potassium phosphate tribasic (1.37 g, 2.1 eq, 6.43 mmol). 2 ml of dry toluene was then added. The reaction mixture was flushed with nitrogen twice. 2-bromonaphtalene (762 mg, 1.2 eq, 3.68 mmol) dissolved in 1.5 ml of dry toluene was added to the reaction mixture followed by the Trans, N,N′-dimethyl-1, 2, cyclohexanediamine (987.2 mg, 0.20 eq, 0.613 mmol) dissolved in 1 mL of dry toluene. The reaction is flushed twice with nitrogen and degassed for 5 minutes. The reaction is sealed and heated at 110° C. for 24 hours.

The reaction is allowed to cool down to room temperature. The crude reaction mixture was dissolved in EtOAc and filtered through celite. The celite was washed three times with EtOAc, concentrated in vacuo.

Purification by column chromatography (Jones 25 g) using 10% EtOAc/90% petroleum gave 282 mg of isomer A, and 53 mg of isomer B.

¹H NMR (CDCl₃) Isomer A: 8.67 (1H, q, J 0.81 Hz), 8.31 (1H, d, J 0.87 Hz), 8.08 (1H, t, J 0.88 Hz), 8.05 (1H, t, J 0.93 Hz), 8.01 (1H, d, J 8.79 Hz), 7.89 (3H, m), 7.80 (1H, dd, J 3.63 Hz), 7.53 (2H, m, 3.11 Hz).

¹H NMR (CDCl₃) Isomer B: 8.75 (1H, t, J 0.91 Hz), 8.60 (1H, d, J 0.84 Hz), 8.33 (1H, s), 7.99 (2H, d, J 1.83 Hz), 7.89 (3H, m), 7.79 (1H, dd, J 3.24 Hz), 7.53 (2H, q, J 2.95 Hz).

### Step 2: Nitro Reduction

### Illustrated for Conversion of B to D.

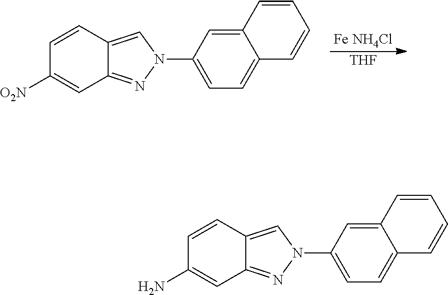

Isomer A or B (120 mg, 1 eq, 0.415 mmol) and ammonium chloride (45 mg, 2 eq, 0.830 mmol) were dissolved in a mixture of THF (4 mL)/H₂O (1 mL) and the mixture was heated at 80° C. Iron powder (116 mg, 5 eq, 2.07 mmol) was then added to the mixture. The reaction was heated for 12 hours at 80° C. No starting material was left y TLC. The reaction mixture was allowed to cool down to room temperature and filtered through celite. The celite was washed three times with THF, concentrated in vacuo. The residue was dissolved in EtOAc, washed twice with H₂O and once with brine. The organic phases were combined, dried over sodium sulfate and concentrated in vacuo. No purification attempted

### Step 3: Amide Formation

### Illustrated for Conversion of D to F.

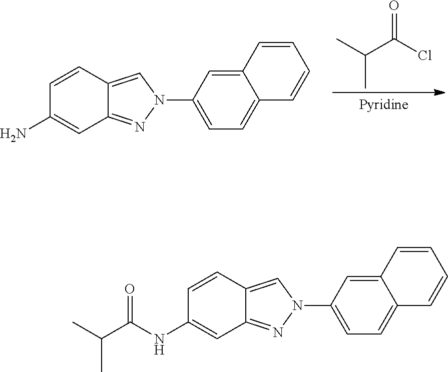

The crude aniline C or D (130 mg, 1 eq, 0.5 mmol) was dissolved in pyridine (8 mL) and the isobutyrylchloride was added drop wise at room temperature. The reaction mixture was left stirring at room temperature for 18 hours.

No starting material was observed by TLC after 18 hours. The reaction mixture was diluted with CuSO_{4 aq}, extracted three times with EtOAc. The combined organic phases were washed once with brine and H₂O, dried over sodium sulfate and concentrated in vacuo.

Purification by column chromatography (Jones 2 g) using 20% EtOAc/80% Petroleum following by trituration in ether to remove any trace of solvent.

Isomer A: red solid LCMS RT=6.88 M+1=330.2

Isomer B: white solid LCMS RT=6.42 M+1=330.2

¹H NMR (CDCl₃) Isomer E: 8.51 (1H, s), 8.17 (2H, dd, J 3.75 Hz), 8.02 (1H, d, J 8.76 Hz), 7.93 (3H, td, J 4.25 Hz), 7.73 (1H, d, J 8.58 Hz), 7.54 (2H, m, J 2.13 Hz), 7.43 (1H, NH, s), 7.09 (1H, dd, J 3.45 Hz), 2.56 (1H, m, J 6.87 Hz), 1.29 (6H, d, J 6.87 Hz).

¹H NMR (CDCl₃) Isomer F: 8.41 (1H, s), 8.28 (1H, s), 7.96 (2H, m, J 5.60 Hz), 7.86 (3H, m, J 5.03 Hz), 7.60 (1H, d, J 8.94 Hz), 7.48 (2H, m, J 2.75 Hz), 7.19 (1H, NH, s), 7.17 (1H, s), 2.50 (1H, m, J 6.77 Hz), 1.23 (6H, d, J 6.77 Hz).

### Example 2

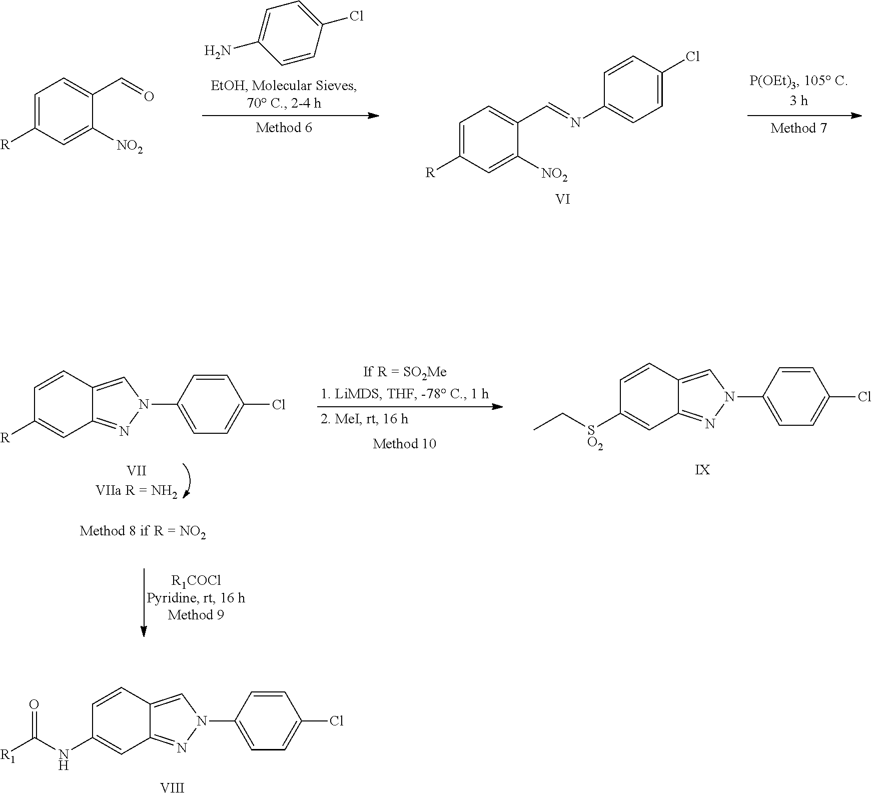

### 2-(4-Chlorophenyl)-6-(ethylsulfonyl)-2H-indazole

To a dry schlenk under nitrogen was added 2-(4-chlorophenyl)-6-(methylsulfonyl)-2H-indazole (200 mg, 0.65 mmol) and dry tetrahydrofuran (14 mL). The solution was then cooled down to −78° C., and lithium bis(trimethylsilyl)amide (0.65 mL, 0.65 mmol) was added. The reaction was left stirring at −78° C. for 2 h, and then methyl iodide (81 μL, 1.31 mmol) was added. The solution was allowed to warm up to room temperature for 16 h. Aqueous saturated ammonium chloride (10 mL) was added to the solution, the organic layer was separated and the aqueous layer was extracted three times with ethyl acetate. The combined organic layers were dried over anhydrous MgSO₄ and evaporated. The resulting solid was purified by column chromatography eluting with ethyl acetate/hexanes 1:2 v/v, combined with a previous batch and 108 mg were purified by reverse phase HPLC to afford 85 mg of the title compound (LCMS RT=6.39 min, MH⁺322.2)

¹H NMR (DMSO): 9.37 (1H, d, J 0.5 Hz), 8.32 (1H, d, J 0.8 Hz), 8.20 (2H, d, J 8.9 Hz) 8.09 (1H, dd, J 8.9, 0.6 Hz), 7.74 (2H, d, J 8.9 Hz), 7.54 (1H, dd, J 8.8 1.5 Hz), 3.40 (2H, q, J 7.3 Hz), 1.15 (3H, t, J 7.4 Hz).

### Example 3

### 5-(Ethylsulfonyl)-2-(naphthalen-2′-yl)-2H-benzo[d][1,2,3]triazole

To a dry schlenk under nitrogen was added 5-(methylsulfonyl)-2-(naphthalen-2-yl)-2H-benzo[d][1,2,3]triazole (83 mg, 0.26 mmol) and dry tetrahydrofuran (8 mL). The solution was then cooled down to −78° C., and lithium bis(trimethylsilyl)amide (0.28 mL, 0.28 mmol) was added. The reaction was left stirring at −78° C. for 1 h, and then methyl iodide (33 μL, 0.52 mmol) was added. The solution was allowed to warm up to room temperature for 16 h. Aqueous saturated ammonium chloride was added to the solution, the organic layer was separated and the aqueous layer was extracted twice with ethyl acetate. The combined organic layers were dried over anhydrous MgSO₄ and evaporated. The resulting solid was purified by column chromatography eluting with ethyl acetate/hexanes 1:2 v/v followed by another column chromatography eluting with ethyl acetate/hexanes 1:3 v/v and then purified by reverse phase HPLC to afford 19 mg (22%) of the title compound (LCMS RT=7.25 min, (2M+NH₄)⁺692.0); ¹H NMR (DMSO): 9.02 (1H, d, J 2.1 Hz), 8.70-8.71 (1H, m), 8.53 (1H, dd, J 8.9 2.2 Hz), 8.39 (1H, dd, J 9.0 0.8 Hz), 8.27-8.30 (2H, m), 8.11-8.14 (1H, m), 7.98 (1H, dd, J 9.0 1.6 Hz), 7.69-7.73 (2H, m). 3.48 (2H, q, J 7.3 Hz), 1.19 (311, t, J 7.3 Hz).

1. A compound of formula (Ia) or (Ib): 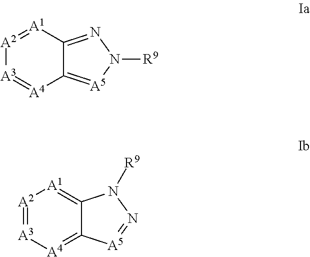
wherein
three of A¹, A², A³, and A⁴ represent CH and one of A¹, A², A³, and A⁴ represents CR¹;
A⁵ represents CH or N;
R¹ represents SO₂R² or NHCOR² wherein R² represents C₁-C₆ alkyl optionally substituted by one or more halogen, hydroxyl or alkoxy groups;
R⁹ represents C₆-C₁₀ aryl, optionally substituted by one or more halogen groups;
or a pharmaceutically acceptable salt thereof.

2. The compound of claim 1 having formula Ia.

3. The compound of claim 1, wherein R⁹ represents 2-naphthyl or 4-chlorophenyl.

4. The compound of claim 1, wherein A¹, A² and A⁴ represent CH and A³ represents CR¹.

5. The compound of claim 1, wherein R² is ethyl or isopropyl.

6. The compound selected from the following table of claim 1, wherein the compound is:
N-(1-(naphthalen-2-yl)-1H-indazol-6-yl)isobutyramide;
N-(2-(naphthalen-2-yl)-2H-indazol-6-yl)isobutyramide;
2-(4-chlorophenyl)-6-(ethylsulfonyl)-2H-indazole; or
5-(ethylsulfonyl)-2-(naphthalen-2′-yl)-2H-benzo[d][1,2,3]triazole.

7. A pharmaceutical composition, comprising a compound of claim 1 and a pharmaceutically acceptable carrier.

8. A method for the treatment or prophylaxis of Duchenne muscular dystrophy, Becker muscular dystrophy or cachexia, comprising administering the compound of claim 1.

9. (canceled)
